(19) United States
(12) Patent Application Publication
(10) Pub. No.: US 2025/0114128 A1
(43) Pub. Date: Apr. 10, 2025

(54) TRANSVERSE CONNECTOR

(71) Applicant: Zavation Medical Products, LLC, Flowood (MS)

(72) Inventors: Noah Carlton Slack, Jackon, MS (US); John Lawrence Walker, Madison, MS (US); John Franklin Cummins, Kosciusko, MS (US)

(21) Appl. No.: 18/481,880

(22) Filed: Oct. 5, 2023

(51) Int. Cl. A61B 17/70

(52) U.S. Cl. CPC A61B 17/705; A61B 17/701

(57) ABSTRACT

A transverse connector for interconnecting first and second spine rods. The connector has a first connecting member connectable with the first spine rod, a second connecting member connectable with the second spine rod and fixed a set distance from the first connecting member, and a coupler integrally attached to only one of the first and second connecting members, and configured to angularly couple the first and second connecting members together such that an angle of deployment between the first connecting rod and the second connecting rod is adjustable. The connector has a fastener of the coupler which is configured to fix the angle of deployment in any one of a plurality of angular positions.

### BACKGROUND OF THE INVENTION

### Field of the Invention

The present invention relates to implant devices for treating in particular spinal disorders.

### Description of the Related Art

Disorders of the bone include disorders such as for example degenerative disc disease, disc herniation, osteoporosis, spondylolisthesis, stenosis, scoliosis (and other curvature abnormalities), kyphosis, tumor, fracture, arthritis, calcification, etc. Such disorders may result from factors including trauma, disease and degenerative conditions caused by injury and aging. Bone disorders typically result in symptoms including pain, nerve damage, and partial or complete loss of mobility. Additionally, severe pain and discomfort can occur due to the pressure exerted by bones on nerves.

A spinal disc can become damaged as a result of degeneration, dysfunction, disease and/or trauma. Conservative treatment can include non-operative treatment through exercise and/or pain relievers to deal with the pain. Operative treatment options include disc removal and replacement using interbody spacers such as anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF).

The spacers are placed in the interdiscal space between adjacent vertebrae of the spine, resulting in spinal fusion of the adjacent vertebra wherein two or more vertebrae are joined together (fused) by way of interbody spacers, sometimes with bone grafting, to form a single bone. The current standard of care for interbody fusion requires surgical removal of all or a portion of the intervertebral disc. After removal of the intervertebral disc, the interbody spacer is inserted in the space between the adjacent vertebrae.

Ideally, the interbody spacer should stabilize the intervertebral space and allow fusion of the adjacent vertebrae. Moreover, during the time it takes for fusion to occur, the interbody spacer body should have sufficient structural integrity to withstand the stress of maintaining the space without substantially degrading or deforming and have sufficient stability to remain securely in place prior to actual bone ingrowth fusion.

To assist in stabilization, various methods and support systems can be used in orthopedic surgery to maintain a spatial relationship between multiple bones or bone fragments. For example, various conditions of the spine, such as fractures, deformities, and degenerative disorders, can be treated by attaching a spinal fixation system to one or more vertebrae.

As described in U.S. Pat. No. 10,966,761 (the entire contents of which are incorporated herein by reference), there are many instances in which it may be desirable to connect multiple implants to each other. Yet, there can be various difficulties associated with connecting multiple implants to each other. The available space for the implanted construct can often be very limited, particularly in the cervical area of the spine. Also, aligning and positioning implants and connectors in the surgical wound may be challenging or cumbersome for the surgeon.

As detailed in the '761 patent, a procedure for stabilization can begin by forming an open or percutaneous incision in the patient to access a target site. The target site can be one or more vertebrae, a long bone or multiple portions of a long bone, or any other bone or non-bone structure of the patient. As shown in FIG. 1 reproduced from the '761 patent, the target site can be multiple vertebrae in a patient's cervical and thoracic spine.

Bone anchors can be driven into one or more of the vertebrae and spinal rods can be attached thereto using known techniques. Bilateral spinal rods R1, R2 can be coupled to four adjacent vertebrae V1-V4 using eight bone anchors S1-S8. In addition, bilateral rods R3, R4 can be coupled to two additional vertebrae V5-V6 using four bone anchors S9-S12. The rods R1, R2 can be connected to the rods R3, R4, respectively, using two connectors C1-C2 of the type described herein.

As detailed in the '761 patent, connectors C1-C2 can be articulated and locked in an articulated position as shown. This can allow the principal longitudinal axes of the rods R1, R3 to be obliquely angled with respect to each other, and/or for the principal longitudinal axes of the rods R2, R4 to be obliquely angled with respect to each other.

### SUMMARY OF THE INVENTION

In one embodiment, there is provided a transverse connector for interconnecting first and second spine rods. The connector has a first connecting member connectable with the first spine rod, a second connecting member connectable with the second spine rod and fixed a set distance from the first connecting member, and a coupler integrally attached to only one of the first and second connecting members, and configured to angularly couple the first and second connecting members together such that an angle of deployment between the first connecting rod and the second connecting rod is adjustable. The connector has a fastener of the coupler which is configured to fix the angle of deployment in any one of a plurality of angular positions.

In another embodiment, there is provided a system for stabilizing a spinal column using the above-noted transverse connector.

In another embodiment, there is provided a method for stabilizing a spinal column using the above-noted transverse connector.

It is to be understood that both the foregoing general description of the invention and the following detailed description are exemplary, but are not restrictive of the invention.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing, wherein:

FIG. 1 is a e depiction of a prior art system for stabilizing a spinal column;

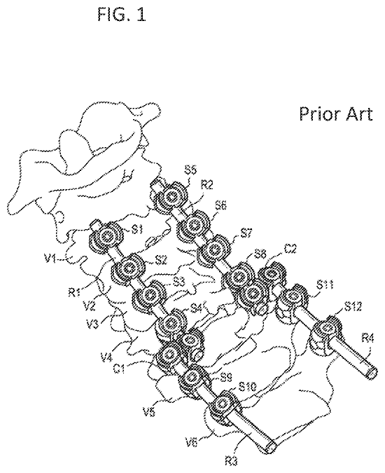

FIG. 2 is a depiction of a transverse connector according to embodiments of the present invention;

FIG. 3 is another view of the transverse connector shown in FIG. 2;

FIG. 4 is a sectional view of the transverse connector shown in FIG. 2;

FIG. 5 is still another view of the transverse connector shown in FIG. 2;

FIG. 6 is a different transverse connector of the present invention;

FIG. 7 is a depiction of a system for stabilizing a spinal column according to embodiments of the present invention; and

FIG. 8 is a flowchart depicting a method for stabilization of vertebrae according to embodiments of the present invention.

### DETAILED DESCRIPTION OF THE INVENTION

Embodiments of the disclosure are generally directed to intervertebral implant devices. One or more fixation members, such as screws, nails, shims, tangs, spikes, staples, pins, fins, blades, or the like, may be used to secure the implant devices to adjacent vertebrae. The fixation members may also include a combination of these to provide for optimal ease of insertion and fixation of the device. The intervertebral implant devices may be included with an inserter for placing the implant devices into the intervertebral space and securing the implant devices to the vertebrae with the fixation members.

The embodiments of the disclosure and the various features and advantageous details thereof are explained more fully with reference to the non-limiting embodiments and examples that are described and/or illustrated in the accompanying drawings and detailed in the following description. The features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

Terms such as distal and proximal are used herein to designate the relative positions of components in relation to a particular view of the object designated, with proximal representing a part of the object closer and distal representing a part of the object farther removed from the proximal. These terms are used for clarity in description, but the examples and embodiments herein should not be construed to mean that the invention does not cover the same objects when viewed from a different perspective. Furthermore, in some cases (but not all), these terms may be synonymous with human body reference terms anterior and posterior.

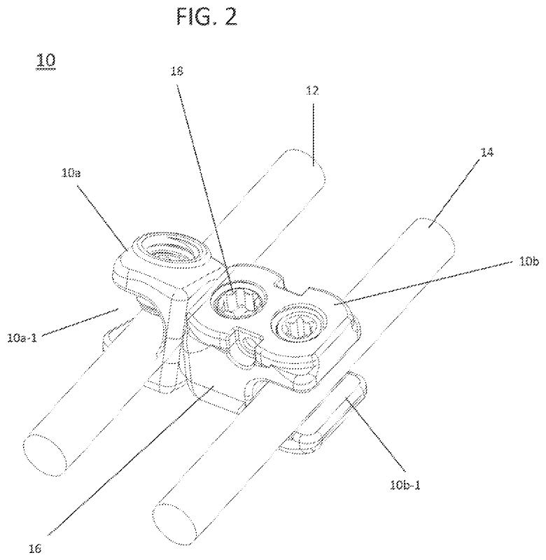

In one embodiment of the present invention, as shown in FIG. 2, a transverse connector 10 is provided for interconnecting first and second spine rods 12, 14. The connector 10 has a) a first connecting member 10a connectable with the first spine rod 12, b) a second connecting member 10b connectable with the second spine rod 14, and c) a coupler 16 configured to angularly couple the first and second connecting members 10a, 10b together. The first connecting member 10a and the second connecting member 10b have respective transverse openings 10a-1 and 10b-1 for insertion of the first and second spine rods 12 and 14. The connector has a fastener 18 for fixing the angle of deployment in any one of a plurality of pivot positions.

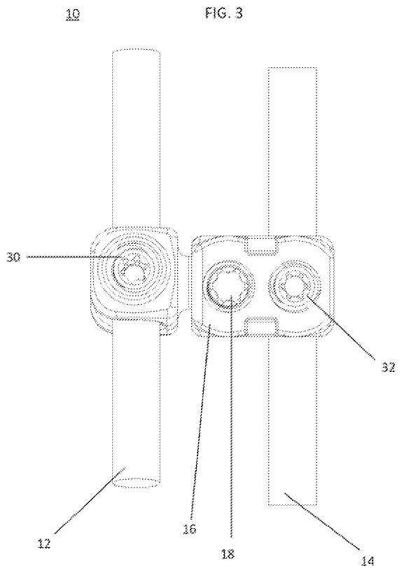

As illustrated in FIGS. 2 and 3, an angle of deployment is adjustable between the first connecting rod 12 and the second connecting rod 14 in at least two complementary angular directions.

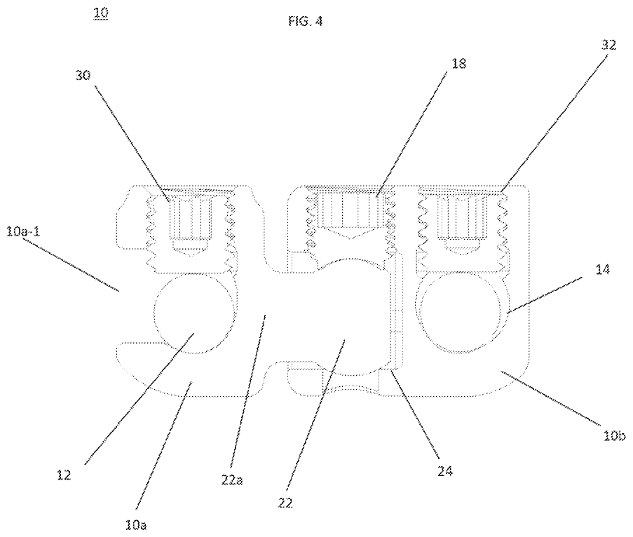

As seen in FIG. 4, coupler 16 has a ball 22 on one end of the first connecting member 10b which engages in a socket 24 on the second connecting member 10a. In one embodiment, the coupler is integrally attached to one of the first connecting member 10b or the second connecting member 10a. The ball 22 may comprise a fixed length arm 22a (in one embodiment) integrally attached to the first connecting member 10b. The present invention is not limited to having the above-noted elements integrally attached. As illustrated, the arm 22a extends toward the coupler 16. Fastener 18 may comprise a set screw extending through the socket 24 and engaging the ball 22 of the ball joint to fix the angle of deployment. The first and second connecting members 10a, 10b are configured to clamp respectively to the first and second spine rods 12, 14. For example, screws 30, 32 engage threaded apertures in first and second connecting members 10a, 10b. The screws 30, 32 when advanced inward engage the first and second spine rods 12, 14 and thereby fixes the first connecting member 10a to the first spine rod 12 and fixes the second connecting member 10b to the second rod 15 to prevent translation of the first and second spine rods relative to the first and second connecting members 10a, 10b.

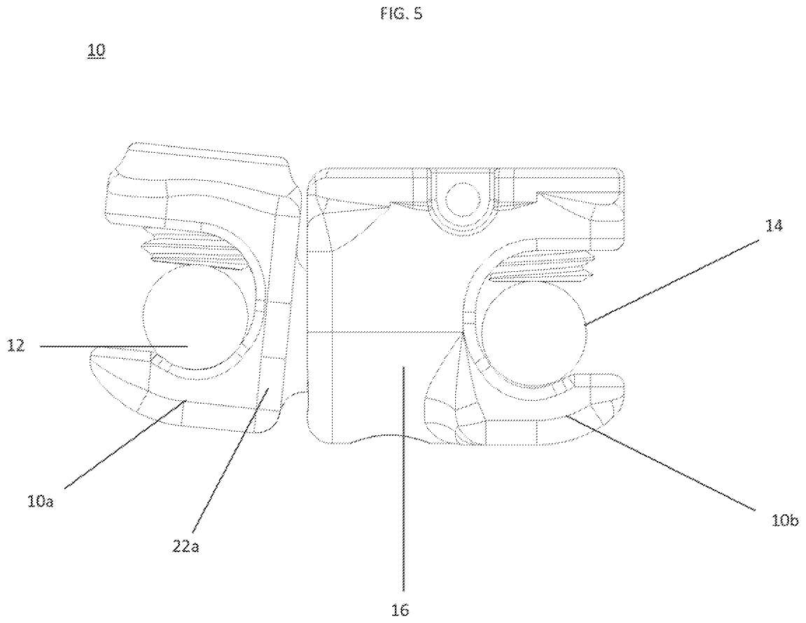

The ball and ball joint construction permits additional angular adjustments as seen in FIG. 5 showing a rotation changing the angle between the first and second connecting members 10a, 10b. In general, the coupler 16 with its ball and ball joint construction permits angular variations all around a longitudinal axis of the first connecting member 10a. In one embodiment, the coupler 16 does not allow for lateral adjustments. Furthermore, coupler 16 is not configured to attach to spinal rods disposed on opposite sides of the spine.

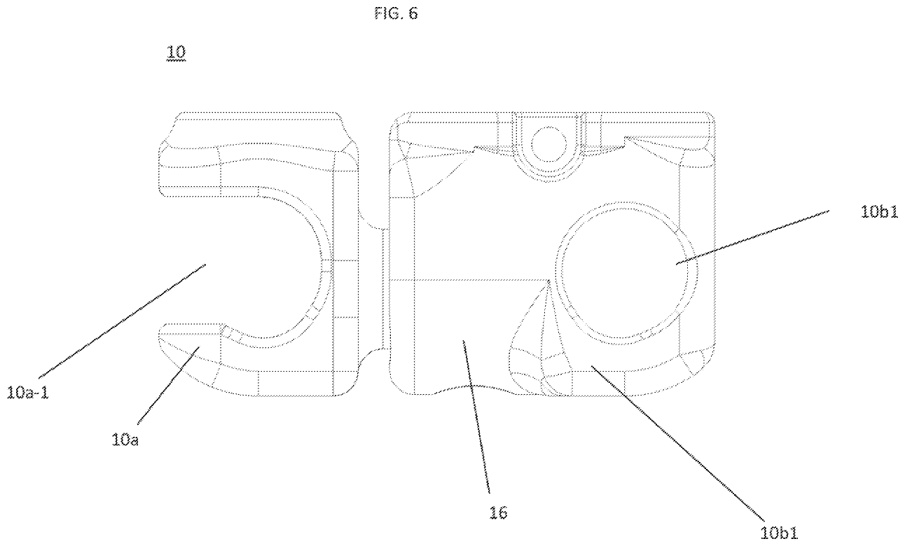

In another embodiment, the first and second connecting members 10a, 10b may have openings 10a-1 and 10b-1 as shown in FIG. 6 where one of the openings is closed ended and the other is open-ended. In one embodiment, both openings 10a-1 and 10b-1 could be closed ended or open-ended.

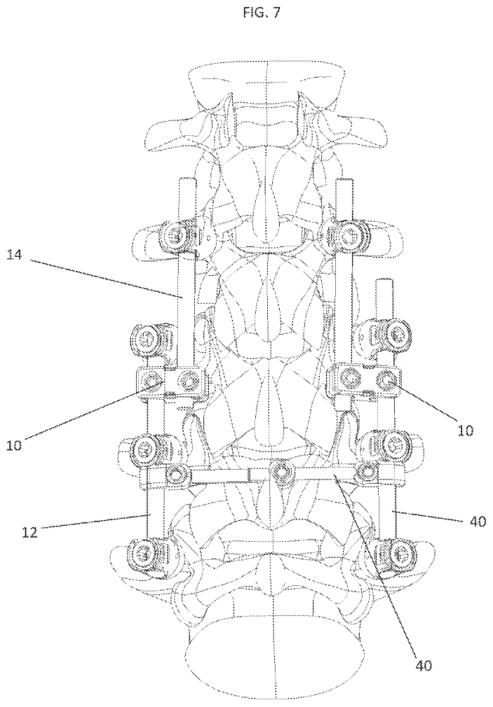

In another embodiment of the present invention, there is provided a system for stabilizing a spinal column, as illustrated in FIG. 7. In FIG. 7, transverse connectors 10 are used on each side of the spinal column to connect rods such as rods 12. 14 extending along one side of the spinal column. In addition, as illustrated in FIG. 7, lateral connectors 40 extend across the spinal column and attach to rods on each side of the spinal column. This system stabilizes the spinal column using in addition to the lateral connectors the transverse connectors 10 of the present invention as described above. This system includes a first spine rod 12, a second spine rod 14, a first connecting member 10a connectable with the first spine rod 12, a second connecting member 10b connectable with the second spine rod 14 and fixed a set distance from the first connecting member 10a, and a coupler 16 (in one embodiment) integrally attached to only one of the first and second connecting members, disposed between the first and second connecting members, and configured to angularly couple the first and second connecting members together such that an angle of deployment is adjustable between the first connecting rod and the second connecting rod. This system includes a fastener 18 for fixing the angle of deployment in any one of a plurality of angularly diverse positions, and one or more lateral connectors 40 extending across the spinal column to connect to rods 42, 44 on the other side of the spinal column.

In one embodiment of this system, the first connecting member comprises a ball having a fixed length arm integrally attached to the first connecting member, and the arm extends toward the coupler disposed between the first and second connecting members.

In one embodiment of this system, the coupler comprises a ball joint for engaging the ball of the first connecting member, and the fastener of the coupler can engage the ball of the ball joint to fix the angle of deployment.

In one embodiment of this system, the fastener comprises a screw engages the ball of the ball joint. In one embodiment of this system, the first connecting member and the second connecting member have respective transverse openings for insertion of the first and second spine rods, and the first and second connecting members can clamp respectively the first and second spine rods in the transverse opening.

In one embodiment of this system, the coupler permits angular variations around a longitudinal axis of the transverse connector. In one embodiment of this system, the the first connecting member comprises a first threaded aperture through which a first set screw, upon advancing through the threaded aperture, fixes the first connecting member to the first spine rod. In one embodiment of this system, the second connecting member comprises a second threaded aperture through which a second set screw, upon advancing through the second threaded aperture, fixes the second connecting member to the second rod.

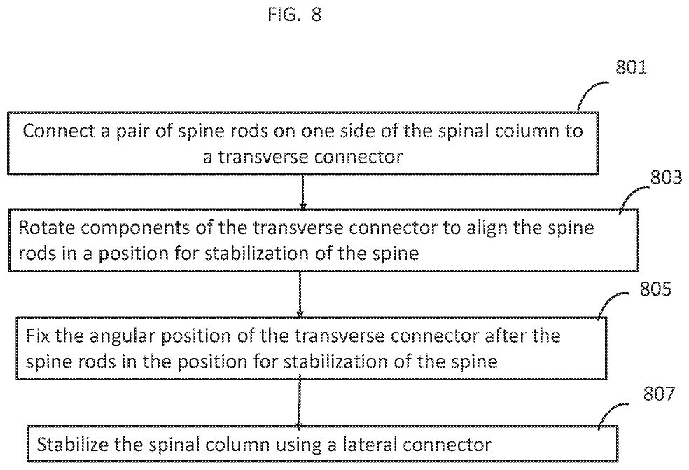

In another embodiment, there is provided a method for stabilization of vertebrae. FIG. 8 is a flowchart depicting this method for stabilization of vertebrae. This method at 801 connects a pair of spine rods on one side of the spinal column to a transverse connector.

At 803, rotate components of the transverse connector to align the spine rods in a position for stabilization of the spine.

At 805, fix the angular position of the transverse connector after the spine rods in the position for stabilization of the spine.

At 807, optionally stabilize the spinal column using a lateral connector connecting the pair of spine rods to another set of rods across the spinal column from the pair of spine rods.

This invention is also not limited to the type of material that the components of the transverse connector 10 is made from. The transverse connector 10 can be made of any material appropriate for human implantation and having the mechanical properties sufficient to be utilized for the intended purpose of spinal stabilization, including various metals such as cobalt chrome, stainless steel or titanium including its alloys, various plastics including those which are bio-absorbable, and various ceramics or combination sufficient for the intended purpose.

This invention is also not limited to the methods by which the transverse connector 10 are made. The individual components can be machined from solid stock pieces. Molding can be used to make the individual components. In this case, machining to final dimensions may or may not be in order. The surfaces once properly dimensioned can be coated with a variety of biocompatible coatings and/or surface treatments.

In some embodiments, any of the components described above can be used with additional implants and instruments. In some embodiments, the implants and instruments can be used with other stabilization members, such as plates and screws securing the spine rods in position to specific vertebrae along the spinal column.

### STATEMENTS OF THE INVENTION

The following are non-limiting statements of the invention describing various aspects of the invention.

Statement 1. A transverse connector for interconnecting first and second spine rods, comprising:
a first connecting member connectable with the first spine rod;
a second connecting member connectable with the second spine rod and fixed a set distance from the first connecting member;
a coupler integrally attached to only one of the first and second connecting members, and configured to angularly couple the first and second connecting members together such that an angle of deployment between the first connecting rod and the second connecting rod is adjustable; and
a fastener of the coupler configured to fix the angle of deployment in any one of a plurality of angular positions.

Statement 2. The connector of statement 1, wherein
the first connecting member comprises a ball having a fixed length arm integrally attached to the first connecting member, and
the arm extends toward the coupler.

Statement 3. The connector of any of the above statements, wherein the coupler comprises a ball joint for engaging the ball of the first connecting member.

Statement 4. The connector of any of the above statements, wherein the fastener of the coupler engages the ball of the ball joint to fix the angle of deployment.

Statement 5. The connector of any of the above statements, wherein the fastener comprises a screw engages the ball of the ball joint.

Statement 6. The connector of any of the above statements, wherein the first connecting member and the second connecting member have respective transverse openings for insertion of the first and second spine rods.

Statement 7. The connector of any of the above statements, wherein the first and second connecting members clamp respectively the first and second spine rods in the transverse opening.

Statement 8. The connector of any of the above statements, wherein the coupler permits angular variations around a longitudinal axis of the transverse connector.

Statement 9. The connector of any of the above statements, wherein the first connecting member comprises a first threaded aperture through which a first set screw, upon advancing through the threaded aperture, fixes the first connecting member to the first spine rod.

Statement 10. The connector of any of the above statements, wherein the second connecting member comprises a second threaded aperture through which a second set screw, upon advancing through the second threaded aperture, fixes the second connecting member to the second rod.

Statement 11. A system for stabilizing a spinal column, comprising:
a first spine rod;
a second spine rod;
a first connecting member connectable with the first spine rod;
a second connecting member connectable with the second spine rod and fixed a set distance from the first connecting member;
a coupler integrally attached to only one of the first and second connecting members, disposed between the first and second connecting members, and configured to angularly couple the first and second connecting members together such that an angle of deployment is adjustable between the first connecting rod and the second connecting rod;
a fastener for fixing the angle of deployment in any one of a plurality of angularly diverse positions; and
one or more lateral connectors extending across the spinal column to connect to rods on the other side of the spinal column.

Statement 12. The system of statement 11, wherein
the first connecting member comprises a ball having a fixed length arm integrally attached to the first connecting member, and
the arm extends toward the coupler disposed between the first and second connecting members.

Statement 13. The system of any of the above statements after statement 11, wherein the coupler comprises a ball joint for engaging the ball of the first connecting member.

Statement 14. The system of any of the above statements after statement 11, wherein the fastener of the coupler engages the ball of the ball joint to fix the angle of deployment.

Statement 15. The system of any of the above statements after statement 11, wherein the fastener comprises a screw engages the ball of the ball joint.

Statement 16. The system of any of the above statements after statement 11, wherein the first connecting member and the second connecting member have respective transverse openings for insertion of the first and second spine rods.

Statement 17. The system of any of the above statements after statement 11, wherein the first and second connecting members clamp respectively the first and second spine rods in the transverse opening.

Statement 18. The system of any of the above statements after statement 11, wherein the coupler permits angular variations around a longitudinal axis of the transverse connector.

Statement 19. The system of any of the above statements after statement 11, wherein the first connecting member comprises a first threaded aperture through which a first set screw, upon advancing through the threaded aperture, fixes the first connecting member to the first spine rod.

Statement 20. The system of any of the above statements after statement 11, wherein the second connecting member comprises a second threaded aperture through which a second set screw, upon advancing through the second threaded aperture, fixes the second connecting member to the second rod.

Statement 21. A method for interconnecting first and second spine rods using any of the connectors of statements 1-10, the method comprising:
angularly coupling first and second connecting members together such that an angle of deployment between a first connecting rod and a second connecting rod is adjustable; and
fixing the angle of deployment in any one of a plurality of angular positions.

Statement 22. A method for stabilizing a spinal column using any of the systems of statements 11-20, the method comprising:
angularly coupling first and second connecting members together such that an angle of deployment is adjustable between a first connecting rod and a second connecting rod;
fixing the angle of deployment in any one of a plurality of angularly diverse positions; and
connecting one or more lateral connectors to rods on the other side of the spinal column.

Statement 23. The method of statement 22, comprising:
connecting a pair of spine rods on one side of the spinal column to a transverse connector;
rotating components of the transverse connector to align the spine rods in a position for stabilization of the spine;
fixing the angular position of the transverse connector after the spine rods in the position for stabilization of the spine; and
stabilizing the spinal column using a lateral connector.

Statement 24. A transverse connector for interconnecting first and second spine rods on one side of a spinal column, comprising:
a first connecting member connectable with the first spine rod;
a second connecting member connectable with the second spine rod;
a coupler comprising a ball and arm attached to one of the first and second connecting members, and configured to angularly couple the first and second connecting members together such that an angle of deployment between the first connecting rod and the second connecting rod is adjustable; and
a fastener of the coupler configured to fix the angle of deployment in any one of a plurality of angular positions by engaging the ball.

Statement 25. A transverse connector for interconnecting first and second spine rods on one side of a spinal column, comprising:
a first connecting member connectable with the first spine rod;
a second connecting member connectable with the second spine rod;
a coupler attached to one of the first and second connecting members; and
a fastener of the coupler configured to fix an angle of deployment between the first connecting rod and the second connecting rod
wherein the first connecting member comprises a ball having a fixed length arm attached to the first connecting member, and
the arm extends toward the coupler.

Statement 26. A transverse connector for interconnecting first and second spine rods on one side of a spinal column, comprising:

a first connecting member connectable with the first spine rod;

a second connecting member connectable with the second spine rod;

a coupler of fixed length attached to one of the first and second connecting members and providing angular variations around a longitudinal axis of the coupler; and

a fastener of the coupler configured to fix an angle of deployment between the first connecting rod and the second connecting rod.

Statement 27. A transverse connector for interconnecting first and second spine rods on one side of a spinal column, comprising:
a first connecting member connectable with the first spine rod;
a second connecting member connectable with the second spine rod;
a coupler attached to one of the first and second connecting members; and
a fastener of the coupler configured to fix an angle of deployment between the first connecting rod and the second connecting rod.

Statement 28. A method for interconnecting first and second spine rods on one side of a spinal column using any of the connectors of statements 24-27, the method comprising:
angularly coupling first and second connecting members together such that an angle of deployment between a first connecting rod and a second connecting rod is adjustable; and
fixing the angle of deployment in any one of a plurality of angular positions.

Statement 29. A system for interconnecting first and second spine rods on one side of a spinal column using any of the connectors of statements 24-27.

Statement 30. A system for stabilizing a spinal column, comprising:
a first spine rod;
a second spine rod;
a first connecting member connectable with the first spine rod;
a second connecting member connectable with the second spine rod;
a coupler attached to one of the first and second connecting members, disposed between the first and second connecting members, and configured to angularly couple the first and second connecting members together such that an angle of deployment is adjustable between the first connecting rod and the second connecting rod;
a fastener for fixing the angle of deployment in any one of a plurality of angularly diverse positions; and
one or more lateral connectors extending across the spinal column to connect to rods on the other side of the spinal column.

Statement 31. A system for stabilizing a spinal column, comprising:
a first spine rod;
a second spine rod;
a first connecting member connectable with the first spine rod;
a second connecting member connectable with the second spine rod;
a coupler, of fixed length attached to one of the first and second connecting members and providing angular variations around a longitudinal axis of the coupler;
a fastener for fixing the angle of deployment in any one of a plurality of angularly diverse positions; and
one or more lateral connectors extending across the spinal column to connect to rods on the other side of the spinal column.

Statement 32. A system for stabilizing a spinal column, comprising:
a first spine rod;
a second spine rod;
a first connecting member connectable with the first spine rod;
a second connecting member connectable with the second spine rod;
a coupler comprising a ball and arm attached to one of the first and second connecting members, and configured to angularly couple the first and second connecting members together such that an angle of deployment between the first connecting rod and the second connecting rod is adjustable
a fastener for fixing the angle of deployment in any one of a plurality of angularly diverse positions; and
one or more lateral connectors extending across the spinal column to connect to rods on the other side of the spinal column.

Numerous modifications and variations of the present invention are possible in light of the above teachings. It is therefore to be understood that within the scope of the appended claims, the invention may be practiced otherwise than as specifically described herein.

1. A transverse connector for interconnecting first and second spine rods, comprising:
a first connecting member connectable with the first spine rod;
a second connecting member connectable with the second spine rod and fixed a set distance from the first connecting member;
a coupler integrally attached to only one of the first and second connecting members, and configured to angularly couple the first and second connecting members together such that an angle of deployment between the first connecting rod and the second connecting rod is adjustable; and
a fastener of the coupler configured to fix the angle of deployment in one of a plurality of angular positions.

2. The connector of claim 1, wherein
the first connecting member comprises a ball having a fixed length arm integrally attached to the first connecting member, and
the arm extends toward the coupler.

3. The connector of claim 2, wherein the coupler comprises a ball joint for engaging the ball of the first connecting member.

4. The connector of claim 2, wherein the fastener of the coupler engages the ball of the ball joint to fix the angle of deployment.

5. The connector of claim 4, wherein the fastener comprises a screw engages the ball of the ball joint.

6. The connector of claim 1, wherein the first connecting member and the second connecting member have respective transverse openings for insertion of the first and second spine rods.

7. The connector of claim 1, wherein the first and second connecting members clamp respectively the first and second spine rods in the transverse opening.

8. The connector of claim 1, wherein the coupler permits angular variations around a longitudinal axis of the transverse connector.

9. The connector of claim 1, wherein the first connecting member comprises a first threaded aperture through which a first set screw, upon advancing through the threaded aperture, fixes the first connecting member to the first spine rod.

10. The connector of claim 9, wherein the second connecting member comprises a second threaded aperture through which a second set screw, upon advancing through the second threaded aperture, fixes the second connecting member to the second rod.

11. A system for stabilizing a spinal column, comprising:
a first spine rod;
a second spine rod;
a first connecting member connectable with the first spine rod;
a second connecting member connectable with the second spine rod and fixed a set distance from the first connecting member;
a coupler integrally attached to only one of the first and second connecting members, disposed between the first and second connecting members, and configured to angularly couple the first and second connecting members together such that an angle of deployment is adjustable between the first connecting rod and the second connecting rod;
a fastener for fixing the angle of deployment in any one of a plurality of angularly diverse positions; and
one or more lateral connectors extending across the spinal column to connect to rods on the other side of the spinal column.

12. The system of claim 11, wherein
the first connecting member comprises a ball having a fixed length arm integrally attached to the first connecting member, and
the arm extends toward the coupler disposed between the first and second connecting members.

13. The system of claim 12, wherein the coupler comprises a ball joint for engaging the ball of the first connecting member.

14. The system of claim 12, wherein the fastener of the coupler engages the ball of the ball joint to fix the angle of deployment.

15. The system of claim 14, wherein the fastener comprises a screw engages the ball of the ball joint.

16. The system of claim 11, wherein the first connecting member and the second connecting member have respective transverse openings for insertion of the first and second spine rods.

17. The system of claim 11, wherein the first and second connecting members clamp respectively the first and second spine rods in the transverse opening.

18. The system of claim 11, wherein the coupler permits angular variations around a longitudinal axis of the transverse connector.

19. The system of claim 11, wherein the first connecting member comprises a first threaded aperture through which a first set screw, upon advancing through the threaded aperture, fixes the first connecting member to the first spine rod.

20. The system of claim 19, wherein the second connecting member comprises a second threaded aperture through which a second set screw, upon advancing through the second threaded aperture, fixes the second connecting member to the second rod.
